(19) United States
(12) Patent Application Publication
(10) Pub. No.: US 2019/0348184 A1
(43) Pub. Date: Nov. 14, 2019

(54) TUMOR TRACKING WITH INTELLIGENT TUMOR SIZE CHANGE NOTICE

(71) Applicant: KONINKLIJKE PHILIPS N.V., EINDHOVEN (NL)

(72) Inventors: Merlijn SEVENSTER, Haarlem (NL); Michael Stephen HODGE, San Francisco, CA (US)

(73) Assignee: KONINKLIJKE PHILIPS N.V., EINDHOVEN (NL)

(21) Appl. No.: 16/466,673

(22) PCT Filed: Dec. 1, 2017
(86) PCT No.: PCT/EP2017/081106

§ 371 Date: Jun. 5, 2019

Related U.S. Application Data
(60) Provisional application No. 62/429,894, filed on Dec. 5, 2016.

(51) Int. Cl. G16H 70/20; G16H 15/00; G16H 30/40; G16H 50/20

(52) U.S. Cl. CPC G16H 70/20; G16H 50/20; G16H 30/40; G16H 15/00

(57) ABSTRACT

A tumor tracking device (110) includes a guideline engine (130), a detection engine (132), and a user interface (134). The guideline engine (130) receives a current measurement and a plurality of prior measurements of at least one lesion according to a medical image of a subject, each of the current and the plurality of prior measurements identified chronologically, and calculates growth between the current measurement and a most recent measurement of the plurality of prior measurements. The detection engine (132) calculate growth between the current measurement and each of non-most recent measurements of the plurality of prior measurements, and identified at least one of the non-most recent measurements in response to calculated growth between the current measurement and each of non-most recent measurements of the plurality of prior measurements exceeding a threshold according to a medical guideline and the calculated growth between the current measurement and a most recent measurement of the plurality of prior measurements failing to exceed the threshold. The user interface (134) displays on a display device (136) an indicator (138) of the identified at least one of the non-most recent measurements of the at least one lesion.

### FIELD OF THE INVENTION

The following generally relates to medical imaging and cancer treatment and more specifically to longitudinal tracking of lesion measurements in medical images from medical imaging modalities, such as computed tomography (CT) reconstruction, positron emission tomography (PET), single photon emission computed tomography (SPECT), magnetic resonance (MR), and combinations thereof.

### BACKGROUND OF THE INVENTION

Interpretation of medical images can include quantitative measurements, such as the dimensions of a lesion. For example, in Response Evaluation Criteria In Solid Tumors (RECIST), a measurement of a mass lesion is taken across the lesion at a long diameter, and a measurement of a lymph node lesion is taken across the lesion at a short diameter. Both diameters are often measured, particularly for undifferentiated lesions. Guidelines, such as RECIST or those from the World Health Organization (WHO) establish standards of care in evaluating the measured tumors.

For example, RECIST guidelines call for imaging examinations and evaluations of tumor growth according to type and schedule of treatment, 3-4 months after treatment, or 6-8 weeks for phase II trials. The evaluation of tumor growth compares lesions in current examination E(0) with a most recent examination E(−1) and identifies those tumors with more than a 20% threshold growth in diameter as a progressive disease (PD) and those less than a 20% threshold growth in diameter as stable disease (SD). The World Health Organization (WHO) uses a 30% threshold. The selection of the short diameter or the long diameter for comparison is according to the pathology of the lesion, which is either the mass or the lymph node. According to formal guidelines, the disease status of the tumor is typically then used to determine treatment options for a patient.

One issue with current oncological practice is the possible presence of “creep,” which is the appearance of the lesion as a “stable disease” between any two consecutive examinations, but unequivocally growing in a larger time span. For example, between consecutive examinations, a lesion is measured as less than 20% growth according to RECIST guidelines, yet the lesion is still growing.

Healthcare professionals, such as a radiologist, receive the medical images for patients and interpret or read the images, which includes measuring the lesions, classifying the pathology, and preparing a report. Healthcare professionals are under time pressures to interpret the medical images and prepare reports, which can be used by other healthcare professionals, such as oncologists, to treat patients, but are expected to adhere to standards of care. That is, healthcare professional can exceed the standards of care, however, they should also meet and be consistent with the standards of care.

### SUMMARY OF THE INVENTION

Aspects described herein address the above-referenced problems and others.

The following describes longitudinal tumor tracking that is consistent with and exceeds guidelines for tumor tracking. A tumor tracking device and methods include notice of intelligent longitudinal comparisons that consider other growth characteristics of prior examinations, such as lesions growth from each non-most recent prior measurement, growth according to functions of prior measurements, growth adjusted for timing of prior measurements, undifferentiated pathology, and combinations thereof. In some embodiments, a pathology of an undifferentiated lesion is derived from clinical description of a tracked lesion. In some embodiments, the notice is generated for a medical report.

In one aspect, a tumor tracking device includes a guideline engine, a detection engine, and a user interface. The guideline engine receives a current measurement and a plurality of prior measurements of at least one lesion according to a medical image of a subject, each of the current and the plurality of prior measurements identified chronologically, and calculates growth between the current measurement and a most recent measurement of the plurality of prior measurements. The detection engine calculate growth between the current measurement and each of non-most recent measurements of the plurality of prior measurements, and identified at least one of the non-most recent measurements in response to calculated growth between the current measurement and each of non-most recent measurements of the plurality of prior measurements exceeding a threshold according to a medical guideline and the calculated growth between the current measurement and a most recent measurement of the plurality of prior measurements failing to exceed the threshold. The user interface displays on a display device an indicator of the identified at least one of the non-most recent measurements of the at least one lesion.

In another aspect, a method of tumor tracking includes receiving a current measurement and a plurality o f prior measurements of at least one lesion according to a medical image of a subject, each of the current and the plurality of prior measurements identified chronologically. Growth between the current measurement and a most recent measurement of the plurality of prior measurements is calculated. Growth between the current measurement and each of non-most recent measurements of the plurality of prior measurements is calculated. At least one of the non-most recent measurements is identified in response to calculated growth between the current measurement and each of non-most recent measurements of the plurality of prior measurements exceeding a threshold according to a medical guideline and the calculated growth between the current measurement and a most recent measurement of the plurality of prior measurements failing to exceed the threshold. An indicator of the identified at least one of the non-most recent measurements of the at least one lesion is displayed on a display device.

In another aspect, a non-transitory computer-readable storage medium carrying instructions controls one or more processors to receive a current measurement and a plurality of prior measurements of at least one lesion according to a medical image of a subject, each of the current and the plurality of prior measurements identified chronologically. The processors are further controlled to calculate growth between the current measurement and a most recent measurement of the plurality of prior measurements, and calculate growth between the current measurement and each of non-most recent measurements of the plurality of prior measurements. The processors are further controlled to identify at least one of the non-most recent measurements in response to calculated growth between the current measurement and each of non-most recent measurements of the plurality of prior measurements exceeding a threshold according to a medical guideline and the calculated growth between the current measurement and a most recent measurement of the plurality of prior measurements failing to exceed the threshold. The processors are further controlled to display on a display device an indicator of the identified at least one of the non-most recent measurements of the at least one lesion.

These and other aspects of the invention will be apparent from and elucidated with reference to the embodiment(s) described hereinafter.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates an embodiment of a medical imaging system with a tumor tracking device.

FIG. 2 diagrammatically illustrates an example display of longitudinal tumor measurements with intelligent longitudinal comparisons.

FIG. 3 diagrammatically illustrates an example graphical display of longitudinal tumor measurements with intelligent longitudinal comparisons.

FIG. 4 flowcharts an embodiment of a method of longitudinally tracking tumor measurements with intelligent longitudinal comparisons.

### DETAILED DESCRIPTION OF EMBODIMENTS

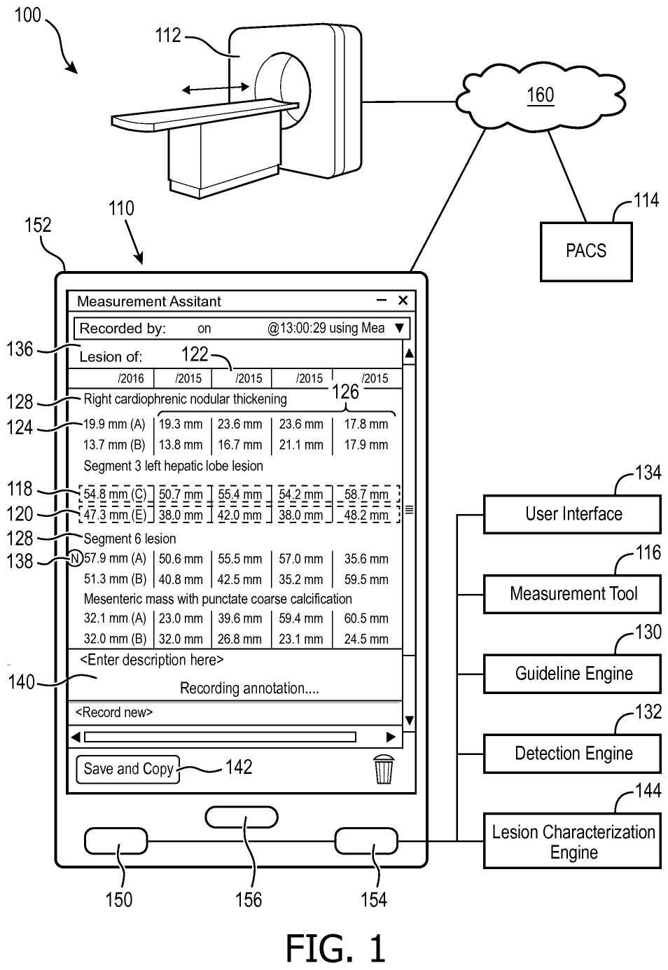

With reference to FIG. 1, an embodiment of a medical imaging system 100 with a tumor tracking device 110 is schematically illustrated. A medical image of a subject can be generated and received directly from a medical imaging scanner 112, such as a computed tomography (CT) scanner, a magnetic resonance (MR) scanner, a positron emission tomography (PET) scanner, single photon emission computed tomography (SPECT) scanner, ultrasound (US) scanner, combinations thereof, and the like. The medical image can be stored in and received from a storage subsystem 114, such as a Picture Archiving and Communication System (PACS), radiology information system (RIS), Electronic Medical Record (EMR), Hospital Information System (HIS) and the like.

A measurement tool 116 can measure lesions in the medical image, such as a long diameter 118 of a lesion, a short diameter 120 of the lesion, and/or both. Measurements are identified chronologically. For example, measurements can include date stamps 122, date/time stamps, and the like. In some embodiments, the date stamp 122 can be retrieved from metadata of an imaging study or examination, such as a Digital Imaging and Communications in Medicine (DICOM) header. Measurements include current measurements 124 according to the medical image or imaging examinations, and prior measurements 126 according to prior medical images or imaging examinations. Measurements are according to each lesion, which can be labeled 128.

A guideline engine 130 receives the current measurement 124 and the prior measurements 126. In some embodiments, the guideline engine 130 receives the current measurement 124 from the measurement tool 116. In some embodiments, the guideline engine 130 receives the current measurement 124 and/or the prior measurements 126 from the storage subsystem 114.

The guideline engine 130 calculates growth between the current measurement 124, E(0), and a most recent measurement, E(−1) of the prior measurements 126 for each measured lesion. For example, a growth rate of a current measurement of 57.9 mm and a most recent measurement of 50.6 mm is 14% ((E(0)−E(−1))/E(−1)), which is less than the threshold of 20% according to RECIST guidelines and is therefore a stable disease (SD). For a mass lesion, measurements of a long diameter are used. For a lymph node, measurements of a short diameter are used. For an undifferentiated lesion, where the type of lesion is unknown, growth rates for each of a long diameter and a short diameter can be calculated. In some embodiments, the growth rate of a measured lesion exceeding the guideline threshold for a progressive disease (PD) is identified. In some instances, this meets the standard of care according to the guideline, which includes notice by an indicator of the PD.

A detection engine 132 calculates growth between the current measurement 124 and characteristics of prior examinations, such as growth from each non-most recent prior examination, growth according to functions of measurements from the prior examinations, growth adjusted for timing of prior examinations, undifferentiated pathology, and/or combinations thereof. In some embodiments, the prior examinations can be limited to a maximum interval from the date of the current measurements. In some embodiments, the prior examinations can be limited according to dates of the current treatment type. In some embodiments, characteristics can be determined for long diameter measurements, short diameter measurements or both measurements. The detection engine identifies at least one of the prior measurements whose characteristic is greater than the guideline threshold for the PD.

A user interface 134 displays on a display device 136 a visual indicator 138 of the identified prior measurement(s) and/or the lesion identified as PD according to the guidelines and combinations thereof. The indicator 138 can include a displayed symbol, a displayed shape, a difference in display intensity of one or more of the plurality of prior measurements, a difference in contrast of one or more of the plurality of prior measurements, a color difference of one or more of the plurality of prior measurements, and combinations thereof. For example, an “N” can indicate a nadir or lowest measurement of the prior measurements exceeds the growth threshold for the corresponding diameter, and the values of the nadir can be highlighted. In another example, a “1” can indicate a first most recent prior measurement exceeding the growth threshold for the corresponding diameter, and the color change for the measurement. In another example, a combination of symbols can include “NR” for growth according to nadir, and “R” for growth adjusted for time. In some embodiments, the indicator includes identification of growth according to guidelines, such as a “G” for guidelines. In some embodiments, no symbols are used, only color changes in the prior measurements. In some embodiments, shapes are used. In some instances, notice by the indicator 138 meets guidelines for PD/SD and exceeds the guidelines by considering other characteristics of a progressive disease, which can include “creep.”

In some embodiments, the display on the display device 136 can include the measured lesions with measurements ordered chronologically or longitudinally as illustrated in the example display of FIG. 1. The lesions can include a label 128, which can be entered or annotated in a character entry area 140. The display on the display device 136 can include the dates of the examinations 122.

The user interface 134, in response to an input 142, can generate a report, which includes the indicator 138. The generated report can include the information about the measurements according to the guidelines, such as the label 128, current 124 and most recent prior measurement 126 of any lesion, which exceeds the threshold for progressive disease (PD) according to the guideline. The format can be the same as the display or different. For example, the report can be formatted as text. An example is “A Mesenteric mass with punctate coarse calcification exceeds RECIST guidelines for progressive diseases with growth from 23.0 mm on Dec. 7, 2015 to 32.1 mm on Feb. 7 2016, which is a 40% growth.” The generated report can include the information about the other growth characteristics. An example is “A segment 6 lesion is indicated as SD according to RECIST guidelines, which is less than 20% growth of 50.6 mm from a Dec. 7, 2015 examination to 59.9 mm of a Feb. 7, 2016 examination. However, the lesion does exhibit growth of 63% over a nadir of 35.6 mm measured in the examination of Jun. 7, 2015.”

A lesion characterization engine 144 can derive a lesion pathology from a clinical description or label 128 of the lesion. For example, if the corresponding label for a lesion includes the word “mass”, then the pathology can be designated as mass for the lesion. Terms used to describe the lesion, which are different for mass lesions and lymph node lesions can be used to indirectly derive the pathology. The guideline engine 130 and the detection engine 132 can use the derived pathology to determine which of the set of measurements, e.g. long diameter or short diameter, to calculate growth for the guidelines and further characteristics.

The measurement tool 116, the guideline engine 130, the detection engine 132, the user interface 134, and the lesion characterization engine 144 are suitably embodied by one or more configured processors, such as one or more processors 150 of a computing device 152. The configured processor(s) 150 execute at least one computer readable instruction stored in computer readable storage medium, such as the memory 154 of the computing device 152, which excludes transitory medium and includes physical memory and/or other non-transitory medium to perform the disclosed lesion measurement, guideline evaluation, growth calculation, measurement identification and indication, lesion characteristic derivation, and display techniques. The configured processor may also execute one or more computer readable instructions carried by a carrier wave, a signal or other transitory medium. The computing device 152 suitably embodies the tumor tracking device 110 and can comprise a workstation, laptop, tablet, smart phone, body worn computing device, server, combinations and the like. The lines between components represented in the diagram represent communications paths, which can be wired or wireless through one or more communication networks 160.

The computing device 150 includes the display device 136, such as a computer display, projector, body worn display, and the like, and one or more input devices 156, such as a mouse, keyboard, microphone, touch or gesture interface, and the like. The computing device 152 includes the one or more processors 150, such as a digital processor, a microprocessor, an electronic processor, an optical processor, a multi-processor, a distribution of processors including peer-to-peer or cooperatively operating processors, client-server arrangement of processors, and the like.

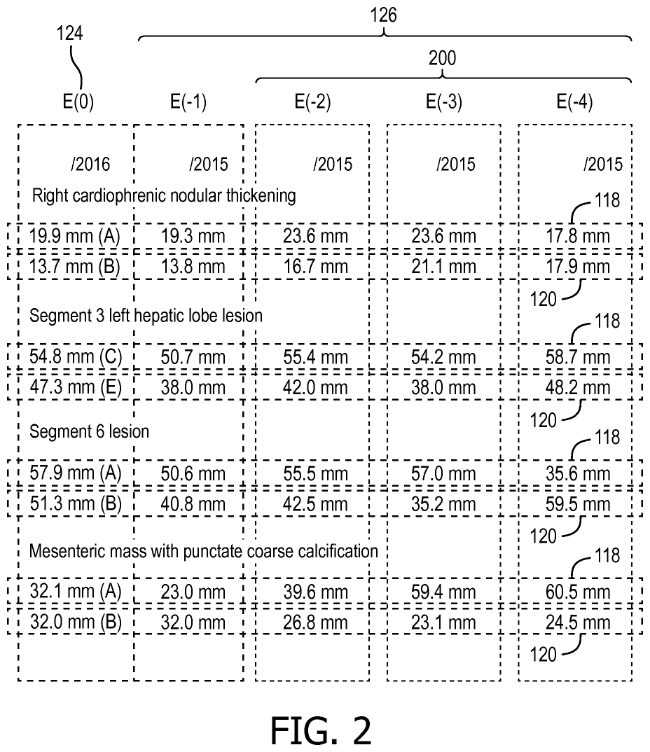

With reference to FIG. 2, an example display of longitudinal tumor measurements with intelligent longitudinal comparisons is diagrammatically illustrated. In some embodiments, the detection engine 132 calculates growth between the current measurement 124 and each of non-most recent measurements 200, E(−2) to E(−N), of the prior measurements 126. For example, with the “Segment 6 lesion” growth is calculated between 55.5 mm long diameter 118 of E(−2) and 59.9 mm of E(0), 57.0 mm long diameter 118 of E(−3) and 59.9 mm of E(0), and 35.6 mm long diameter 118 of E(−4) and 59.9 mm of E(0); and between 40.8 mm short diameter 120 of E(−2) and 51.3 mm of E(0), 35.2 mm short diameter 120 of E(−3) and 51.3 mm of E(0), and 59.5 mm short diameter 120 of E(−4) and 51.3 mm of E(0). Growth of each calculation is compared with the guideline threshold, such as 20% according to RECIST, which identifies E(−4) showing characteristic growth in the long diameter, and E(−2) and E(−3) showing characteristic growth in the short axis for the undifferentiated lesion.

In some embodiments, the detection engine 132 calculates growth between the current measure 124 and a function of the prior measurements 126, such as a minimum, a mean, a median, and the like. For example, growth is calculated between a minimum of (E(−1) to E(−N)) and the current measurement 124. In some instances, the calculated growth according to a minimum represents a comparison with a nadir or lowest point. Using the prior measurements of the short diameter 120 “Segment 6 lesion”, which are 40.8, 42.5, 35.2 and 59.5, the minimum or nadir is identified as 35.2 mm of E(−3), and growth from the nadir to the current measurement 124 of 51.3 mm is 46%.

In some embodiments, the detection engine 132 adjusts the calculated growth between the current measurement 124 and each of the prior measurements 126 according to the chronology of the measurements. For example, if the time period, according to the guideline/treatment type and schedule, is 60 days, and a current measurement is as of Feb. 7, 2016 and a prior measurement is Dec. 28, 2015, then the actual period between examinations is 41 days instead of 60 days. Using growth of 50.6 mm of E(−1) to 57.9 mm of E(0) of long diameter 118 of the “Segment 6 lesion”, growth according to the guideline engine 130 is 14% or SD. However, adjusting the rate by 60/41, the growth is adjusted to 21%, which exceeds the guideline threshold. In some instances, the adjustment according to the date or days between examinations can illustrate characteristic growth of the lesion.

The detection engine 132 can select a most recent of the identified prior measurements 126 or of the identified non-most recent measurements 200 to be indicated. The user interface 134 can display the indicator 138 for the selected most recent of the identified prior measurements 126 or at least one of the non-most recent measurements. For example, in the example above of E(−2) and E(−3) showing characteristic growth in the short axis for the undifferentiated lesion, the most recent of the E(−2) and E(−3) is E(−2). In some instances, the indicator 138 indicating only the selected most recent can identify the most recent growth showing relevant characteristic growth.

In some embodiments, characteristic growth is only indicated for measurements of a long diameter 118 or a short diameter 120 for which guideline growth indicates as SD.

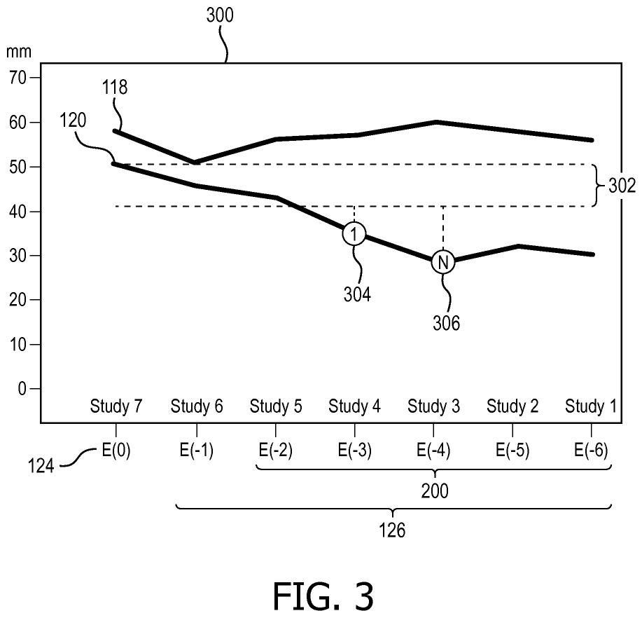

With reference to FIG. 3, an example graphical display of longitudinal tumor measurements with intelligent longitudinal comparisons 300 is diagrammatically illustrated. The example graphical display includes a horizontal axis of examinations or studies ordered chronologically, and a vertical axis of lesion measurements in millimeters (mm). The user interface 134 can display graphically by date of the examinations. The user interface 134 can display graphically the current measurements 124 and a plurality of prior measurements 126 according to the chronology, such as a line graph, bar chart, scatter plot, and the like. The display can include the long diameter measurements 118, the short diameter measurements 120, or both. The display can include the indicator 138 of the identified characteristic growth.

The display can include a threshold indicator 302, which indicates the range of growth, which exceeds the guideline threshold. In the example graphical display 300, the threshold indicator 302 is illustrated as two broken lines extending from the current measurement 124 of the short diameter measurements 120, that is, 20% growth to the current measurement of 50.7 mm. The threshold indicator 302 can alternatively be included with different colors, intensities, symbols, combinations thereof, and the like.

Indicators 138 are illustrated as an identified first non-most recent measurement 304 and an identified nadir 306. The identified first non-most recent measurement 304 is first among the non-most recent measurements 200 with growth calculated to the current measurement 124 exceeding the threshold. In the example display, the most recent measurement, E(−1) of the prior measurements 126 is used for calculated growth by the guideline engine 130. The growth between the remaining of the prior measurements 126 or non-most recent measurements, E(−2), E(−3), E(−4), E(−5), and E(−6), are compared with the threshold, and the growth between each of (E(−3), E(−4), E(−5), E(−6)) and the current measurement 124 are identified as greater than the threshold. That is the calculated growth between the current measurement 124 and each of (E(−3), E(−4), E(−5), E(−6)) are identified as greater than the threshold. Chronologically, E(−3) is selected as the first measurement of (E(−3), E(−4), E(−5), E(−6)) with calculated growth greater than the threshold.

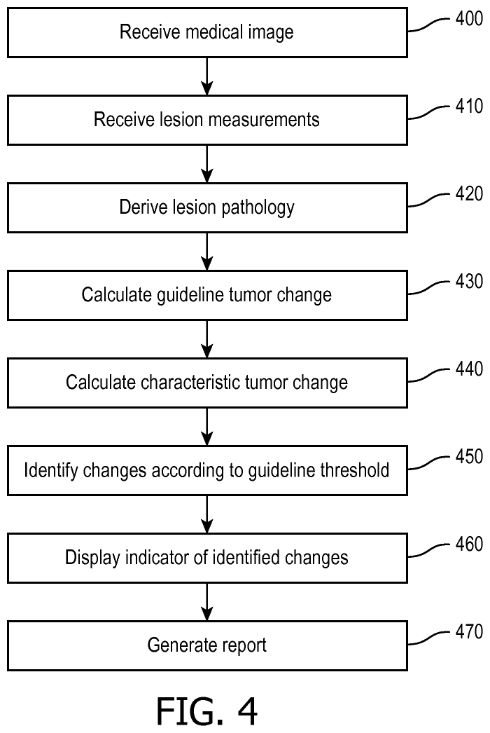

With reference to FIG. 4, an embodiment of a method of longitudinally tracking tumor measurements with intelligent longitudinal comparisons is flowcharted.

At 400, a medical image of a subject can be received. The medical image includes lesions that are longitudinally tracked. The medical image can be received directly from the medical imaging scanner 112 or from the storage subsystem 114.

At 410, the current measurements 124 and prior measurements 126 of lesions are received. The measurements can include the long diameter 118, the short diameter 120 or both for each measured lesion. The measurements include the label 128 or description of each lesion. The measurements include a chronology of the measurements, such as a date stamp of the examination from which a measurement was obtained. The current measurements 124 and the prior measurements 126 can be received from the storage subsystem 114. The current measurements 124 can be received by direct measurement of the medical image, such as by the measurement tool 116.

A pathology of one or more lesions can be derived at 420 from the label 128 or description of each lesion where pathology is undifferentiated. The derivation can include matching of one or more terms or combinations of terms indicative of and specific to a mass pathology or indicative of and specific to a lymph node pathology.

At 430, growth is calculated between the current measurement 124, E(0), and a most recent measurement, E(−1), of the prior measurements 126.

At 440, characteristic growth is calculated. The characteristic growth can include growth between the current measurement 124 and each of the non-most recent measurements 200. The characteristic growth can include growth between the current measurement 124 and a function of the non-most recent measurements 200. The characteristic growth can include growth adjusted for time between the current measurement 124 and one or more of the prior measurements 126. For example, a guideline growth rate is determined according to the threshold and an expected time interval between examinations.

The adjusted growth can be adjusted by differences between the expected time interval for examinations and the actual time interval. The characteristic growth can include combinations of the above.

At 450, the prior measurements corresponding to the characteristic growth are identified in response to the characteristic growth exceeding the guideline threshold. The characteristic growth can include measurements of lesions that are determined as SD according to the guidelines, and can exclude measurements of lesions that are determined as PD according to the guidelines. For example, the calculated growth corresponding to the non-most recent measurements are indicated in response to calculated growth exceeding the threshold and the calculated growth between the current measurement and a most recent measurement is less than the threshold. The identification can include one or more prior measurements according to differently calculated growth, such as non-most recent growth, functions of prior measurements, and growth adjusted for actual time intervals between examinations.

The indicator 138 of the identified prior measurements is displayed on the display device 136. The indicator can be co-located with current measurement 124 or the prior measurements 126 or the label 128 of the corresponding lesion. The display can include the current measurement 124 and the prior measurements 126, such as illustrated with reference to FIG. 1. The display of the indicator 138 can include a graphical display, such as illustrated with reference to FIG. 3.

A medical report can be generated at 470 in response to an input. The medical report includes the indicator 138, which can be formatted in a tabular format of the textual display, such with reference to FIG. 1, or in a graphical format, such as with reference to FIG. 3, or as text, such as described in reference to FIG. 1.

The above may be implemented by way of computer readable instructions, encoded or embedded on computer readable storage medium, which, when executed by a computer processor(s), cause the processor(s) to carry out the described acts. Additionally or alternatively, at least one of the computer readable instructions is carried by a signal, carrier wave or other transitory medium.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A tumor tracking device, comprising:
a guideline engine comprising one or more processors configured to:
receive a current measurement and a plurality of prior measurements of at least one lesion according to a medical image of a subject, each of the current and the plurality of prior measurements identified chronologically;
calculate growth between the current measurement and a most recent measurement of the plurality of prior measurements; and
a detection engine, comprising the one or more processors configured to:
calculate growth between the current measurement and each of non-most recent measurements of the plurality of prior measurements; and
identify at least one of the non-most recent measurements in response to calculated growth between the current measurement and each of non-most recent measurements of the plurality of prior measurements exceeding a threshold according to a medical guideline and the calculated growth between the current measurement and a most recent measurement of the plurality of prior measurements failing to exceed the threshold; and
a user interface comprising the one or more processors configured to display on a display device an indicator of the identified at least one of the non-most recent measurements of the at least one lesion.

2. The device according to claim 1, wherein the detection engine is further configured to select a most recent of the identified at least one of the non-most recent measurements;
wherein the user interface displays the indicator for the selected most recent of the identified at least one of the non-most recent measurements.

3. The device according to claim 1, wherein the detection engine calculated growth includes growth between the current measurement and a calculated minimum of the plurality of prior measurements.

4. The device according to claim 1, wherein the detection engine is further configured to:
determine a guideline growth rate according to the threshold and an examination interval of the guideline; and
wherein the detection engine adjusts the calculated growth according to the chronology of each of the non-most recent measurements and the determined guideline growth rate.

5. The device according to claim 1, wherein the user interface is further configured to:
display graphically the current and plurality of prior measurements with the indicator of the identified at least one of the non-most recent measurements of the at least one lesion.

6. The device according to claim 1, wherein each of the current measurement and the plurality of prior measurements include a long diameter measurement and a short diameter measurement; and further including:
a lesion characterization engine comprising the one or more processors configured to derive a lesion pathology from a clinical description of the at least one lesion.

7. The device according to claim 1, wherein the user interface is further configured to generate a medical report which includes the indicator and corresponding measurements of the at least one lesion.

8. The device according to claim 1, wherein the indicator includes one or more from the group comprising of a displayed symbol, a displayed shape, a difference in display intensity of one or more of the plurality of prior measurements, a difference in contrast of one or more of the plurality of prior measurements, and a color difference of one or more of the plurality of prior measurements.

9. The device according to claim 1, wherein the user interface is further configured to:
receive a medical image of a subject which includes the at least one lesion and data identifying the subject and chronology from at least one of the group comprising a medical imaging scanner and a medical image storage system; and
display a view of the medical image which includes the at least one lesion; and further including:
a measurement tool configured to measure at least one diameter of the at least one lesion displayed in the view, which generates the current measurement.

10. A medical imaging system, comprising:
the tumor tracking device according to claim 1; and
a medical imaging scanner, configured to scan a subject and generate the medical image.

11. A method of tumor tracking, comprising:
receiving a current measurement and a plurality of prior measurements of at least one lesion according to a medical image of a subject, each of the current and the plurality of prior measurements identified chronologically;
calculating growth between the current measurement and a most recent measurement of the plurality of prior measurements;
calculating growth between the current measurement and each of non-most recent measurements of the plurality of prior measurements;
identifying at least one of the non-most recent measurements in response to calculated growth between the current measurement and each of non-most recent measurements of the plurality of prior measurements exceeding a threshold according to a medical guideline and the calculated growth between the current measurement and a most recent measurement of the plurality of prior measurements failing to exceed the threshold; and
displaying on a display device an indicator of the identified at least one of the non-most recent measurements of the at least one lesion.

12. The method according to claim 11, wherein identifying includes:
selecting a most recent of the identified at least one of the non-most recent measurements; and
wherein displaying includes displaying the indicator for the selected most recent of the identified at least one of the non-most recent measurements.

13. The method according to claim 11, wherein calculating the growth between the current measurement and each of non-most recent measurements of the plurality of prior measurements includes:
determining a guideline growth rate according to the threshold and an examination interval of the guideline; and
adjusting the calculated growth according to the chronology of each of the non-most recent measurements and the determined guideline growth rate.

14. The method according to claim 11, wherein calculating the growth between the current measurement and each of non-most recent measurements of the plurality of prior measurements includes calculating growth between the current measurement and a calculated minimum of the plurality of prior measurements.

15. The method according to claim 11, wherein displaying includes:
displaying graphically the current and plurality of prior measurements with the indicator of the identified at least one of the non-most recent measurements of the at least one lesion.

16. A non-transitory computer-readable storage medium carrying instructions which controls one or more processors to:
receive a current measurement and a plurality of prior measurements of at least one lesion according to a medical image of a subject, each of the current and the plurality of prior measurements identified chronologically;
calculate growth between the current measurement and a most recent measurement of the plurality of prior measurements;
calculate growth between the current measurement and each of non-most recent measurements of the plurality of prior measurements;
identify at least one of the non-most recent measurements in response to calculated growth between the current measurement and each of non-most recent measurements of the plurality of prior measurements exceeding a threshold according to a medical guideline and the calculated growth between the current measurement and a most recent measurement of the plurality of prior measurements failing to exceed the threshold; and
display on a display device an indicator of the identified at least one of the non-most recent measurements of the at least one lesion.

17. The non-transitory computer-readable storage medium according to claim 16, wherein to identify includes selecting a most recent of the identified at least one of the non-most recent measurements; and
wherein to display includes displaying the indicator for the selected most recent of the identified at least one of the non-most recent measurements.

18. The non-transitory computer-readable storage medium according to claim 16, wherein to calculate the growth between the current measurement and each of non-most recent measurements of the plurality of prior measurements includes:
determining a guideline growth rate according to the threshold and an examination interval of the guideline; and
adjusting the calculated growth according to the chronology of each of the non-most recent measurements and the determined guideline growth rate.

19. The non-transitory computer-readable storage medium according to claim 16, wherein to calculate the growth between the current measurement and each of non-most recent measurements of the plurality of prior measurements includes calculating growth between the current measurement and a calculated minimum of the plurality of prior measurements.

20. The non-transitory computer-readable storage according to claim 16, wherein to display includes:
displaying graphically the current and plurality of prior measurements with the indicator of the identified at least one of the non-most recent measurements of the at least one lesion.
